(19) United States
(12) Patent Application Publication
(10) Pub. No.: US 2023/0070582 A1
(43) Pub. Date: Mar. 9, 2023

(54) UTILIZATION OF FGF ACTIVATORS IN CULTURE MEDIA

(71) Applicant: Yissum Research Development Company of the Hebrew University of Jerusalem Ltd., Jerusalem (IL)

(72) Inventors: Yaakov Nahmias, Mevaseret Zion (IL); Muneef Ayyash, Jerusalem (IL); Laura Pasitka, Jersusalem (IL)

(73) Assignee: Yissum Research Development Company of the Hebrew University of Jerusalem Ltd., Jersusalem (IL)

(21) Appl. No.: 17/794,206

(22) PCT Filed: Jan. 20, 2021
(86) PCT No.: PCT/IB2021/050419

§ 371 Date: Jul. 20, 2022

Related U.S. Application Data
(60) Provisional application No. 62/963,819, filed on Jan. 21, 2020.

(51) Int. Cl. C12N 5/00; C12N 5/077; A23L 13/00

(52) U.S. Cl. CPC C12N 5/0037; C12N 5/0656; A23L 13/00; C12N 2501/115; C12N 2501/119; A23V 2002/00

(57) ABSTRACT

The present disclosure provides, in part, a cell culture medium comprising a serum-free medium and one or more fibroblast growth factor (FGF) activators, a kit comprising the cell culture medium and instructions for use, methods of growing cells in vitro and of producing a cultured meat using the cell culture medium, and a cultured meat so produced.

### CROSS-REFERENCE TO RELATED APPLICATIONS

This application claims the benefit of priority to U.S. Provisional Patent Application No. 62/963,819, filed on Jan. 21, 2020, which is incorporated herein by reference in its entirety.

### FIELD OF THE INVENTION

The present invention generally relates to cell growth. More specifically, the present invention relates to serum-free cell growth media comprising one or more fibroblast growth factor activators and methods for growing cells in the media, thereby producing cultured meat.

### BACKGROUND

The current world population is over 7 billion and still rapidly growing. In order to support the nutritional requirement of this growing population, an increasing amount of land is dedicated for food production. The natural sources are insufficient to fulfill the demand. This has led to famine in some parts of the world. In other parts of the world, the problem is being addressed by large-scale production of animals in dense factory farms under harsh conditions. This large-scale production not only causes great suffering to animals, but also increases arsenic levels and drug resistance bacteria in meat products due to organoarsenic compounds and antibiotics used to increase food efficiency and control infection, thus further increases the number of diseases and worsens the consequences thereof for both animals and humans. Large-scale slaughtering is required to fulfill the current food requirements and as a consequence, it can lead to large-scale disease outbreaks such as the occurrence of porcine pestivirus and mad cow disease. These diseases result in loss of the meat for human consumption thus completely denying the purpose for which the animals were being bred in the first place.

In addition, the large-scale production reduces the flavor of the finished product. A preference exists among those that can afford non-battery laid eggs and non-battery produced meat. It is not only a matter of taste, but also a healthier choice thereby avoiding consumption of various feed additives such as growth hormones. Another problem associated with mass animal production is the environmental problem caused by the vast amounts of fecal matter from the animals and which the environment subsequently has to deal with. Moreover, the large amount of land currently required for the production of animals or the feed for the animals which cannot be used for alternative purposes such as growth of other crops, housing, recreation, wild nature and forests is problematic.

One of the primary problems of the techniques known in the art is that, with a long time to produce, and at extremely high costs, products are of a mediocre quality that cannot and will not replace the current meat derived from livestock. For example, Just-Inc. grows extracted animal cells in media to manufacture chicken nuggets, which cost $50 per nugget to manufacture.

Culture of cells, e.g., mammalian cells or insect cells, for in vitro experiments or ex vivo culture, for administration to a human or animal is an important tool for studies and treatments of human diseases. Cell culture is widely used for the production of various biologically active products, e.g., viral vaccines, monoclonal antibodies, polypeptide growth factors, hormones, enzymes, tumor specific antigens and food products. However, many of the media or methods used to culture the cells comprise components that can have negative effects on cell growth and/or maintenance of an undifferentiated cell culture. For example, mammalian or insect cell culture media is often supplemented with blood-derived serum such as fetal calf serum (FCS) or fetal bovine serum (FBS) in order to provide growth factors, carrier proteins, attachment and spreading factors, nutrients and trace elements that promote proliferation and growth of cells in culture. However, the factors found in FCS or FBS, such as transforming growth factor (TGF) beta or retinoic acid, can promote differentiation of certain cell types (Ke et al., Am J Pathol. 137: 833-43, 1990) or initiate unintended downstream signaling in the cells that promotes unwanted cellular activity in culture (Veldhoen et al., Nat Immunol. 7(11): 1151-6, 2006).

The cost of culture medium is the primary driving factor of the cost of cultured meat production. Culture medium is composed of relatively simple basal medium that comprises carbohydrates, amino acids, vitamins and minerals and much more expensive serum replacement component including; albumin, growth factors, enzymes, attachment factors and hormones. In order to eliminate the use of animal components, industry is currently relying on recombinant human proteins for applications in cell therapy and vaccine production. However, cultured meat applications are not limited to the use of human proteins, thus can potentially utilize a more readily available source of materials that is suitable for human consumption.

Fibroblast growth factors (FGF) are a family of heparin-binding cell signaling proteins. Members of the FGF family are multifunctional proteins that are involved in a wide variety of biological processes, including embryonic development, organogenesis, cell proliferation, cell migration, cell differentiation and integrin expression (Burgess, et al., Annu Rev Biochem 58, 575-606, 1989; Rifkin, et al., J Cell Biol 109, 1-6, 1989; Basilico et al., Adv Cancer Res 59, 115-165, 1992). In mammals, there are more than 20 different sub-family member-proteins in the FGF super-family, among which 18 sub-family members interact with four signaling tyrosine kinase FGF receptors (FGFR) (Beenken, et al., Nat Rev Drug Discov 8, 235-253, 2009). This interaction leads to downstream activation of several signaling pathways including phospholipase C-γ (PLCγ), phosphatidylinositol-3-kinase (PI3K), and mitogen-activated protein kinase (MAPK) pathways.

FGFs are important mitogens. They are critical components of culture media for biological manufacturing of cell therapies, glycosylated proteins, vaccine and cultured meat. Cost of animal-derived or recombinant human FGF is a significant fraction of the cost of the culture media and a major roadblock in the commercial reality of cultured meat. Moreover, for therapeutic applications and cultured meat, it is preferable to culture the cells in animal-component free (xeno-free) media under chemically defined conditions. Thus, finding small molecules in place of FGFs would provide promising advancement in the field of cultured meat and cell therapy. As such, there is a need for cell culture media without the undesirable side effects of growth or attachment factor serum components. The present disclosure fulfills this long-standing need.

### SUMMARY OF THE INVENTION

The present disclosure is based, in part, on the identification of small molecules that can activate the fibroblast growth factor (FGF) signaling pathway. These small molecules, or FGF activators, include, but are not limited to, PF-05231023 (an FGF21 analog for T2DM), ID-8 (an indole derivative), 1-Azakenpaullone (an activator of the Wnt pathway), Tacrolimus (FK-506) (a macrolide antibiotic), and (E/Z)-BCI hydrochloride (a Dusp6 inhibitor that hyperactivates FGF), or a combination thereof. These long-acting small molecules can replace the effect of FGF in culture media and support cost-effective cell proliferation. The use of such small molecules in culture media significantly reduces the cost of the media for the production of cultured meat.

One aspect of the present disclosure provides a cell culture medium comprising a serum-free medium and one or more fibroblast growth factor (FGF) activators.

In some embodiments, the cell culture medium comprises less than 1 ng/ml of fibroblast growth factor (FGF), epidermal growth factor (EGF), transforming growth factor-β (TGF-β), or a combination thereof.

In some embodiments, the cell culture medium is essentially devoid of any protein-based growth factors excluding peptide-based hormones or steroid-based hormones. In some embodiments, the protein-based growth factors stimulate cell growth and proliferation. In some embodiments, the peptide-based hormone is insulin. In some embodiments, the steroid-based hormone is cortisone or a derivative thereof.

In some embodiments, the one or more FGF activators are one or more small molecules that activate FGF signaling pathway. In some embodiments, the one or more small molecules comprise an FGF21 analog for Type 2 diabetes (T2DM), an indole derivative, an activator of the Wnt pathway, a macrolide antibiotic with an immunosuppressive property, a target of a negative regulator of a downstream pathway of FGF signaling, or a combination thereof.

In some embodiments, the one or more small molecules comprise an FGF21 analog for Type 2 diabetes (T2DM). In some embodiments, the FGF21 analog is PF-05231023.

In some embodiments, the one or more small molecules comprise an indole derivative. In some embodiments, the indole derivative is ID-8.

In some embodiments, the one or more small molecules comprise an activator of the Wnt pathway. In some embodiments, the activator is an inhibitor of glycogen synthase kinase 3β (GSK3β). In some embodiments, the activator is 1-Azakenpaullone.

In some embodiments, the one or more small molecules comprise a macrolide antibiotic with an immunosuppressive property. In some embodiments, the macrolide antibiotic is Tacrolimus (FK-506).

In some embodiments, the one or more small molecules comprise a target of a negative regulator of a downstream pathway of FGF signaling. In some embodiments, the target is an inhibitor that hyperactivates FGF pathway by activating ERK pathway. In some embodiments, the inhibitor is a Dusp6 inhibitor. In some embodiments, the Dusp6 inhibitor is (E/Z)-BCI hydrochloride.

In some embodiments, ID-8 is at a concentration of about 0.5 μM to about 50 μM. In some embodiments, ID-8 is at a concentration of about 1 μM to about 10 μM.

In some embodiments, FK-506 is at a concentration of about 1 nM to about 20 nM. In some embodiments, FK-506 is at a concentration of about 1 nM to about 2 nM.

In some embodiments, the one or more small molecules comprise ID-8 and FK-506.

Still in some embodiments, ID-8 is at a concentration of about 0.5 μM to about 50 μM and FK-506 is at a concentration of about 1 nM to about 20 nM.

In some embodiments, ID-8 is at a concentration of about 1 μM to about 10 μM and FK-506 is at a concentration of about 1 nM to about 2 nM.

Another aspect of the present disclosure provides a kit comprising any of the herein disclosed cell culture medium and instructions for use.

Still another aspect of the present disclosure provides a method of producing a cultured meat by culturing cells in any of the herein disclosed cell culture medium and producing a cultured meat from the cultured cells.

In some embodiments, the cells are from edible animals. In some embodiments, the edible animal is livestock, game, poultry, fish, crustaceans, or mollusk.

In some embodiments, the method comprises cultured cells wherein the cells are fibroblasts. In an embodiment, the fibroblasts are bovine fibroblasts or chicken fibroblasts.

Yet another aspect of the present disclosure provides a cultured meat produced by the methods disclosed above and herein.

Still yet another aspect of the present disclosure provides a method of growing cells in vitro by culturing cells in any of the herein disclosed cell culture medium.

Further aspect of the present invention provides use of one or more FGF activators in a cell culture medium essentially devoid of any protein-based growth factors excluding peptide-based hormones or steroid-based hormones.

### BRIEF DESCRIPTION OF THE DRAWINGS

The foregoing aspects and other features of the invention are explained in the following description, taken in connection with the accompanying drawings, wherein:

FIG. 1 depicts replacement of FGF with ID-8 and/or FK-506 in bovine fibroblasts cultured in serum-free medium suspension.

FIG. 2 depicts replacement of FGF with ID-8 and FK-506 in chicken fibroblasts cultured in serum-free medium suspension.

FIG. 3 depicts replacement of FGF with ID-8 and FK-506 at various concentrations in ovine fibroblasts cultured in serum-free media.

### DETAILED DESCRIPTION OF THE INVENTION

For the purposes of promoting an understanding of the principles of the present disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The terminology used herein is for the purpose of describing particular embodiments only and is not intended to be limiting of the invention.

As used herein, a cell culture medium “devoid of any protein-based growth factor” or “devoid of fibroblast growth factor”, or a cell culture medium “essentially devoid of any protein-based growth factor” or “essentially devoid of fibroblast growth factor”, refers to a medium that does not comprise any detectable amount of protein-based growth factor or fibroblast growth factor (FGF). The term “non-detectable” is understood as based on standard methodologies of detection known in the art at the time of this disclosure. In some embodiments, the medium may comprise less than 1 ng/ml (0 ng/ml to less than 1 ng/ml) protein-based growth factor or FGF. In some embodiments, the medium may comprise less than 0.5 ng/ml (0 ng/ml to less than 0.5 ng/ml) protein-based growth factor or FGF. In some embodiments, the medium may comprise less than 0.1 ng/ml (0 ng/ml to less than 0.1 ng/ml) protein-based growth factor or FGF.

As used herein, “a protein-based growth factor” stimulates cell growth and proliferation, which includes, but is not limited to, FGF, epidermal growth factor (EGF), transforming growth factor-0 (TGF-0), or a combination thereof.

As used herein, a “serum-free” medium refers to a medium that does not contain animal or human serum, in which the components are not derived, obtained, sourced, or produced from animals. It is contemplated that the components are either produced recombinantly or derived from plants or sources other than an animal.

As used herein, “basal media”, “basal medium”, “base media”, “base medium”, “base nutritive medium”, or “base nutritive media” refers to a basal salt nutrient(s) or an aqueous solution(s) of salts and other elements that provide cells with water and certain bulk inorganic ions essential for normal cell metabolism and maintains intra- and extra-cellular osmotic balance. In some embodiments, a base medium comprises at least one carbohydrate as an energy source, and/or a buffering system to maintain the medium within the physiological pH range. Examples of commercially available basal media include, but are not limited to, phosphate buffered saline (PBS), Dulbecco's Modified Eagle's Medium (DMEM), Minimal Essential Medium (MEM), Basal Medium Eagle (BME), RPMI 1640, Ham's F-10, Ham's F-12, α-Minimal Essential Medium (αMEM), Glasgow's Minimal Essential Medium (G-MEM), Iscove's Modified Dulbecco's Medium, or general purpose media modified for use with pluripotent cells, such as X-VIVO (Lonza) or a hematopoietic base media.

As used herein, a “complete medium” refers to a basal medium further comprising added supplements, such as growth factors, hormones, proteins, serum or serum replacement, trace elements, sugars, antibiotics, antioxidants, etc., that can contribute to cell growth. For example, a commercially available complete medium comprises supplements such as ethanolamine, glutathione (reduced), ascorbic acid phosphate, insulin, human transferrin, a lipid-rich bovine serum albumin, trace salts, sodium selenite, ammonium matavanadate, cupric sulfate and manganous chloride (DMEM ADVANCED™ Media, Life Technologies).

As used herein a “liquid base mix” or “base physiological buffer liquid mix” refers to the base liquid solution of the serum replacement or media supplement into which the liposomes are suspended to complete the cell culture media composition. It is contemplated that the liquid base mix is loaded into the liposomes such that the liposome delivers an amount of the liquid base mix to cells when fused to/taken up by cells in cell culture. It is also contemplated herein that the liquid base mix or base physiological buffer liquid mix is a base medium, a complete medium or a physiological buffer solution, such as phosphate buffered saline (PBS) and other balanced salt solutions, which can be used in conjunction with the liposomes and/or other components herein to form a serum replacement, a complete medium, a medium supplement, or a cryopreservation medium.

As used herein, a “medium” or “cell culture medium” refers to an aqueous based solution that provides for the growth, viability, or storage of cells. A medium as contemplated herein can be supplemented with one or more nutrients to promote the desired cellular activity, such as cell viability, growth, proliferation, differentiation of the cells cultured in the medium. A medium, as used herein, includes a serum replacement, a medium supplement, a complete medium or a cryopreservation medium. The pH of a culture medium should be suitable to the microorganisms that will be grown. Most bacteria grow in pH 6.5-7.0 while most animal cells thrive in pH 7.2-7.4.

As used herein, a “medium supplement” refers to an agent or composition that is added to base medium prior to culture of cells. A medium supplement can be an agent that is beneficial to cell growth in culture, such as growth factor(s), hormone(s), protein(s), serum or serum replacement, trace element(s), sugar(s), antibiotic(s), antioxidant(s), etc. Typically, a medium supplement is a concentrated solution of the desired supplement to be diluted into a complete or base medium to reach the appropriate final concentration for cell culture.

As used herein, “serum replacement” or “serum replacement medium” refers to a composition that can be used in conjunction with a basal medium or as a complete medium in order to promote cell growth and survival in culture. Serum replacement is used in basal or complete medium as a replacement for any serum that is characteristically added to medium for culture of cells in vitro. It is contemplated that the serum replacement comprises proteins and other factors for growth and survival of cells in culture. The serum replacement is added to a basal medium prior to use in cell culture. It is further contemplated that a serum replacement may comprise a base medium and base nutrients such as salts, amino acids, vitamins, trace elements, antioxidants, and the like, such that the serum replacement is useful as a serum-free complete medium for cell culture.

As used herein, the term “connective tissue cells” refers to the various cell types that make up connective tissue. For example, connective tissue cells are fibroblasts, cartilage cells, bone cells, fat cells and smooth muscle cells, or a cell type that can be naturally differentiated from a fibroblast. As used herein, the term “natural differentiation” or “naturally differentiated from” is used to refer to a differentiation that occurs in nature and not a trans-differentiation such as one that can be artificially achieved in a laboratory and is not dedifferentiation. A cell type that can be naturally differentiated from a fibroblast includes a chondrocyte, an adipocyte, an osteoblast, an osteocyte, a myofibroblast, a satellite cell, a myoblast and a myocyte. Connective tissue cells are not mesenchymal stem cells (MSCs) or cells derived from MSCs or pluripotent cells.

In some embodiments, a connective tissue cell is selected from the group consisting of a chondrocyte, an adipocyte, an osteoblast, an osteocyte, a myofibroblast, a satellite cell, a myoblast and a myocyte. In some embodiments, a connective tissue cell is selected from the group consisting of an adipocyte, an osteoblast, an osteocyte, a myofibroblast, a satellite cell, a myoblast and a myocyte. In some embodiments, a connective tissue cell is a fibroblast.

As used herein, the phrase “spontaneously immortalized fibroblast” refers to a fibroblast cell which is capable of undergoing unlimited cell division, and preferably also cell expansion, without being subjected to man-induced mutation, e.g., genetic manipulation, causing the immortalization. The spontaneously immortalized fibroblast is non-genetically modified.

As used herein, a “small molecule” is a low molecular weight (<900 daltons) organic compound that may regulate a biological process, with a diameter on the order of about 1 nm. Larger structures such as nucleic acids and proteins, and many polysaccharides are not small molecules, although their constituent monomers (ribo- or deoxyribonucleotides, amino acids, and monosaccharides, respectively) are often considered small molecules.

As used herein, the terms “replace FGF signaling”, “replace FGF”, and “activate FGF signaling pathway” with respect to the one or more small molecules (i.e., FGF activators) are interchangeable in that these activators replace the effect of FGF in the culture medium and support cost-effective cell proliferation.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Disclosed herein, is a cell culture medium that comprises a serum-free medium and one or more fibroblast growth factor (FGF) activators. In some embodiments, the cell culture medium is essentially devoid of any protein-based growth factors excluding peptide-based hormones or steroid-based hormones. Also disclosed herein are methods of culturing cells in the above and herein disclosed cell culture medium and utilizing such cultures for the production of cultured meat. The disclosed cell culture medium disclosed herein can also be used in kits. It was surprisingly discovered that FGF activators can be utilized in cell culture media in place of growth factors to support cost-effective cell proliferation. The use of these FGF activators in culture media significantly reduces the cost of the media for the production of cultured meat.

Among FGF activators, indole derivatives, e.g., ID-8, were shown to support self-renewal of mouse ESCs in serum-free culture (Miyabayashi, et al., Biosci Biotechnol Biochem 72, 1242-1248, 2008), while the combination of ID-8, 1-azakenpaulone, and Tacrlimus were found to support self-renewal of human ESCs in serum-free culture (Yasuda, et al., Nat Biomed Eng 2, 173-182, 2018). ID-8 is a chemical inhibitor of dual-specificity tyrosine phosphorylation-regulated kinase (DYRK). 1-Azakenpaullone is a potent, selective inhibitor of glycogen synthase kinase 3β (GSK3β) and thus activates the Wnt pathway. Tacrolimus (FK-506) is a macrolide antibiotic with immunosuppressive properties. Tacrolimus inhibits calcineurin phosphatase which leads to inhibition of calcium-dependent events, such as interleukin-2 gene transcription, nitric oxide synthase activation, cell degranulation, and apoptosis (Thomson, et al., Ther Drug Monit 17, 584-591, 1995). Another small molecule, PF05231023, was reported to work as an analog of FGF21 (Thompson, et al., J Pharmacokinet Pharmacodyn 43, 411-425, 2016).

Targeting negative regulators of the downstream pathways of FGF signaling is another approach to find small molecules that can replace FGF. One of these inhibitors is (E/Z)-BCI hydrochloride, a Dusp6 inhibitor that hyperactivates FGF pathway by activating ERK pathway had been shown in zebrafish heart model (Molina, et al., Nat Chem Biol 5, 680-687, 2009).

The present disclosure contemplates addition of one or more growth factor activators in cell culture media, e.g., one or more small molecules that replace fibroblast growth factor (FGF) signaling. As such, one aspect of the present disclosure provides a cell culture medium comprising a serum-free medium and one or more FGF activators. In some embodiments, the cell culture medium is essentially devoid of any protein-based growth factors excluding peptide-based hormones or steroid-based hormones. In some embodiments, the one or more FGF activators are one or more small molecules that activate FGF signaling pathway.

Mammals contain 18 FGF types (FGF1-FGF10 and FGF16-FGF23), which have been grouped into six distinct subfamilies based on phylogeny and sequence homology. Four (4) FGFs share a similar internal core and have a characteristically high binding affinity for both heparin and fibroblast growth factor receptors (FGFRs). FGFRs are tyrosine kinase receptors that contain a heparin-binding sequence, three extracellular immunoglobulin-like domains (D1-D3), a hydrophobic transmembrane domain, and a split intracellular tyrosine kinase domain. The mammalian FGFR family consists of four members (FGFR1-FGFR4). The amino acid sequences of the receptors are highly conserved, with differentiation occurring only in their ligand affinity and tissue distribution. Characteristic of FGFRs is the acid box, which is a serine-rich, acidic sequence in the linker between D1 and D2 domains (Beenken, et al., Nat Rev Drug Discov 8, 235-253, 2009). The acid box and D1 domain are thought to play a role in receptor autoinhibition. The D2-D3 fragment is required for ligand specificity and binding. In vertebrates, four genes encode the FGFRs (FGFR1-FGFR4), and undergo alternative splicing in their extracellular domain to produce many varieties of FGFR1-FGFR4 with varying affinities for their ligands.

The FGF signaling cascade is initiated by the binding of FGF ligands to FGFRs. Following FGF binding, a ligand-dependent dimerization event takes place in which a complex is formed that consists of two FGFs, two heparin sulfate chains, and two FGFRs. Each ligand binds to both receptors, and the receptors make contact with one another via a patch on the D2 domain. This facilitates the transphosphorylation of each receptor monomer by an intrinsic tyrosine kinase domain. At least seven phosphorylation sites have been identified for FGFR1 (Tyr¹⁶³, Tyr⁵⁸³, Tyr⁵⁸⁵, Tyr⁶⁵³, Tyr⁶⁵⁴, Tyr⁷³⁰, and Tyr⁷⁶⁶). Phosphotyrosine groups serve as docking sites for adaptor proteins that regulate downstream signaling. The FGF system is associated with several downstream signaling pathways, among which the best understood are the RAS/mitogen-activating protein (MAP) kinase pathway, the phosphoinositide 3 (PI3) kinase/AKT pathway, and the phospholipase C gamma (PLCγ) pathway.

The main downstream pathway associated with FGF signaling is the RAS/MAP kinase pathway. This pathway is implicated during cellular proliferation and differentiation. MAP kinases are serine/threonine-specific protein kinases that act in response to extracellular stimuli and regulate various cellular processes. Examples of MAP kinase effectors include c-Jun N-terminal kinase (JNK), extracellular signal-regulated kinase (ERK), and p38 mitogen-activated kinase. After an FGF ligand binds to its receptor, an integral step in the signaling pathway is the phosphorylation of the tyrosine residues on the docking protein fibroblast growth factor receptor substrate 2 alpha (FRS2α). This permits binding of adaptor proteins that are associated with signal activation. An FRS2 complex consisting of FRS2α, guanine nucleotide exchange factor 2 (GRB2), GRB2-associated binding protein 1 (GAB1), the son of sevenless (SOS), and tyrosine phosphatase (SHP2) is then formed that facilitates activation of the RAS/MAP kinase and also PI3 kinase/AKT pathways.

The PI3 kinase/AKT pathway is associated with cellular survival and cell fate determination. This pathway may also impact cell polarity. Like the RAS/MAP kinase pathway, the PI3 kinase/AKT pathway is initiated when an FRS2 signaling complex forms. GAB1 protein then links activated FGFRs with PI3 kinase. Downstream of PI3 kinase, phosphoinositide-dependent kinase and AKT (an anti-apoptotic protein kinase) are activated.

Another target molecule of activated FGFR is PLCγ. This pathway is activated upon the binding of the PLCγ molecule to the phosphorylated Tyr766 of the receptor. Inositol triphosphate (IP3) and diacylglycerol (DAG) are then generated by the hydrolysis of activated PLCγ. DAG and cytoplasmic calcium released from the endoplasmic reticulum in response to IP3 together activate protein kinase C (PKC). Though it has not been completely elucidated, the PLCγ kinase pathway influences cell morphology, migration, and adhesion.

The below table contains accepted modulators obtainable from Sigma-Aldrich, Inc. and additional information.

TABLE 1
Modulators and Additional Information

| Family Members | FGFR1 | FGFR2 | FGFR3 | FGFR4 | FGFR5 |
|---|---|---|---|---|---|
| Other | Fms-like | BEK | JTK4 | JTK2 | Not Known |
| Names | tyrosine | KSAM | Cek-2 | TKF | |
| | kinase 2 | Cek-3 | flg-2 | | |
| | c-fgr | KGFR | | | |
| | FLG | | | | |
| | FLT2 | | | | |
| | Cek-1 | | | | |
| | c-fgr | | | | |
| Molecular | 91.8 kDa | 92 kDa | 87.7 kDa | 87.9 kDa | 54.5 kDa |
| Weight | | | | | |
| Structural | 822 aa | 821 aa | 806 aa | 802 aa | 504 aa |
| Data | | | | | |
| Isoforms | 18 | 19 | 3 | Not Known | Not Known |
| Species | Human | Human | Human | Human | Human |
| | Mouse | Mouse | Mouse | Mouse | |
| | Rat | Xenopus | | | |
| | Chicken | | | | |
| | Xenopus | | | | |
| Domain | 3 Ig-like C2- | 3 Ig-like C2- | 3 Ig-like C2- | 3 Ig-like C2- | 2 Ig-like C2- |
| Organization | type domains | type domains | type domains | type domains | type domains |
| | Protein kinase | Protein kinase | Protein kinase | Protein kinase | |
| | domain | domain | domain | domain | |
| Phosphorylation | Tyr⁴⁶³ | Tyr⁶⁵⁷ | Tyr⁶⁴⁸ | Tyr⁶⁴³ | Not Known |
| Sites | Tyr⁵⁸³ | | | | |
| | Tyr⁵⁸⁵ | | | | |
| | Tyr⁶⁵³ | | | | |
| | Tyr⁶⁵⁴ | | | | |
| | Tyr⁷³⁰ | | | | |
| | Tyr⁷⁶⁶ | | | | |
| Tissue | Placenta | Brain | Brain | Myoblasts | Mesenchymal |
| Distribution | Brain | Kidney | Kidney | Lung | Kidney |
| | Liver | Skin | Testis | Liver | Brain |
| | Lungs | Lung | | Kidney | Lung |
| | Uterus | Liver | | | |
| Subcellular | Plasma | Plasma | Plasma | Plasma | Plasma |
| Localization | membrane | membrane | membrane | membrane | membrane |
| Binding | FGF2 | FGF1 and | FGF1 | FGF19 | Not known |
| Partners/ | | FGF2 | | | |
| Associated | | | | | |
| Proteins | | | | | |
| Upstream | FGF-1, 2, 4 | FGF-1, 2, 4, 7 | FGF-1, 2, 4, 9 | FGF-1, 2 | FGF-1, 2 |
| Activators | | | | | |
| Downstream | Sprouty 2 | Sprouty 2 | Shp2 | Shp2 | Not Known |
| Activators | Shp2 | Shp2 | PLC-γ | PLC-γ | |
| | PLC-γ | PLC-γ | STAT1 | STAT1 | |
| | STAT1 | STAT1 | STAT3 | STAT3 | |
| | STAT3 | STAT3 | MAPK | MAPK | |
| | MAPK | MAPK | PI3K | PI3K | |
| | PI3K | PI3K | | | |
| Activators | Not Known | Not Known | Not Known | Not Known | |
| Inhibitors | SU5402 | SU5402 | SU5402 | SU5402 | Not Known |
| | (SML0443) | (SML0443) | (SML0443) | (SML0443) | |
| | PD166285 | PD166285 | PD166285 | PD166285 | |
| | (PZ0116) | (PZ0116) | (PZ0116) | (PZ0116) | |
| | PD173074 | PD173074 | PD173074 | PD173074 | |
| | (P2499) | (P2499) | (P2499) | (P2499) | |
| | PD161570 | PD161570 | PD161570 | PD161570 | |
| | (PZ0109) | (PZ0109) | (PZ0109) | (PZ0109) | |
| | PD166866 | PD166866 | PD166866 | PD166866 | |
| | (PZ0114) | (PZ0114) | (PZ0114) | (PZ0114) | |
| Selective | Not Known | Not Known | Not Known | Not Known | Not Known |
| Activators | | | | | |
| Physiological | Cell | Cell | Cell | Cell | Not Known |
| Function | activation | activation | activation | activation | |
| | Chemotactic | Chemotactic | Chemotactic | Chemotactic | |
| | response | response | response | response | |
| | Cell | Cell | Cell | Cell | |
| | proliferation | proliferation | proliferation | proliferation | |
| | Cell | Cell | Cell | Cell | |
| | differentiation | differentiation | differentiation | differentiation | |
| | Embryonic | Embryonic | Embryonic | Embryonic | |
| | patterning | patterning | patterning | patterning | |
| Disease | Pfeiffer | Crouzon | Achondroplasia | Thyroid | Not Known |
| Relevance | syndrome | syndrome | Crouzon | cancer | |
| | Kallmann | Jackson-Weiss | syndrome | | |
| | syndrome | syndrome | Thanatophoric | | |
| | Stem cell | Apert syndrome | dysplasia | | |
| | leukemia | Pfeiffer syndrome | Bladder cancer | | |
| | Lymphoma | Beare-Stevenson | Cervical cancer | | |
| | | cutis gyrata | Craniosynostosis | | |
| | | syndrome | Adelaide typ | | |
| | | | Multiple | | |
| | | | myeloma | | |

Table Abbreviations: PD 161570: 1-Tert-butyl-3-[6-(2,6-dichloro-phenyl)-2-(4-diethylamino-butylamino)-pyrido [2,3-d]pyrimidin-7-yl]urea; PD 166285: 6-(2, 6-dichlorophenyl)-2-[[4-[2-(diethylamino)ethoxy]phenyl]amino]-8-methyl-Pyri do [2, 3-d]pyrimidin-7(8H)-one dihydrochloride; PD 166866: 1-[2-Amino-6-(3,5-dimethyoxy-phenyl)-pyrido [2,3-d]pyrimidin-7-yl]-3-tert-butyl-urea; PD 173074: N-[2-[[4-(diethylamino)butyl]amino-6-(3,5-dimethoxyphenyl)pyrido [2, 3-d]pyrimidin-7-yl]-N′-(1,1-dimethylethyl)-urea; SU5402: 3-[4-Methyl-2-(2-oxo-1,2-dihydro-indol-3-ylidenemethyl)-1H-pyrrol-3-yl]-propionic acid.

Although there are no known activators of the FGF receptors, there are multiple upstream and downstream activators, all of which could be modulated to become FGF activators. Because of the plurality of pathways involved in FGF signaling, the present disclosure contemplates small molecules involved in any of the pathways that mimic FGF activation.

In some embodiments, the one or more small molecules comprise an FGF21 analog for Type 2 diabetes (T2DM), an indole derivative, an activator of the Wnt pathway, a macrolide antibiotic with an immunosuppressive property, a target of a negative regulator of a downstream pathway of FGF signaling, or a combination thereof.

In some embodiments, the one or more small molecules comprise an FGF21 analog for Type 2 diabetes (T2DM). In some embodiments, the FGF21 analog is PF-05231023.

In some embodiments, the one or more small molecules comprise an indole derivative. In some embodiments, the indole derivative is ID-8.

In some embodiments, the one or more small molecules comprise an activator of the Wnt pathway. In some embodiments, the activator is an inhibitor of glycogen synthase kinase 3β (GSK3β). In some embodiments, the activator is 1-Azakenpaullone.

In some embodiments, the one or more small molecules comprise a macrolide antibiotic with an immunosuppressive property. In some embodiments, the macrolide antibiotic is Tacrolimus (FK-506).

In some embodiments, the one or more small molecules comprise a target of a negative regulator of a downstream pathway of FGF signaling. In some embodiments, the target is an inhibitor that hyperactivates FGF pathway by activating ERK pathway. In some embodiments, the inhibitor is a Dusp6 inhibitor. In some embodiments, the Dusp6 inhibitor is (E/Z)-BCI hydrochloride.

The concentration(s) of the one or more small molecules depend(s) on the activity of the small molecule(s). It is within the purview of one of ordinary skill in the art to determine the optimal concentration of the small molecule(s) to add to the media. In some embodiments, the concentration(s) of the small molecule(s) in the media may be about 0.1 nM to about 100 μM, about 1 nM to about 10 μM, or about 10 nM to about 1 μM. In some embodiments, the concentration(s) of the small molecule(s) in the media may be about 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM or 1 mM.

In some embodiments, the one or more small molecules comprise ID-8, FK-506 or a combination thereof. In some embodiments, ID-8 is at a concentration of about 0.5 μM to about 50 μM. In some embodiments, ID-8 is at a concentration of about 0.5 μM to about 40 μM. In some embodiments, ID-8 is at a concentration of about 0.5 μM to about 30 μM. In some embodiments, ID-8 is at a concentration of about 0.5 μM to about 20 μM. In some embodiments, ID-8 is at a concentration of about 0.5 μM to about 10 μM.

In some embodiments, ID-8 is at a concentration of about 1 μM to about 50 μM. In some embodiments, ID-8 is at a concentration of about 1 μM to about 40 μM. In some embodiments, ID-8 is at a concentration of about 1 μM to about 30 μM. In some embodiments, ID-8 is at a concentration of about 1 μM to about 20 μM. In some embodiments, ID-8 is at a concentration of about 1 μM to about 10 μM. In some embodiments, ID-8 is at a concentration of about 1 μM to about 5 μM.

In some embodiments, ID-8 is at a concentration of about 5 μM.

In some embodiments, FK-506 is at a concentration of about 1 nM to about 20 nM. In some embodiments, FK-506 is at a concentration of about 1 nM to about 10 nM. In some embodiments, FK-506 is at a concentration of about 1 nM to about 5 nM. In some embodiments, FK-506 is at a concentration of about 1 nM to about 2 nM.

In some embodiments, FK-506 is at a concentration of about 1 nM.

In some embodiments, ID-8 is at a concentration of about 0.5 μM to about 10 μM and FK-506 is at a concentration of about 1 nM to about 2 nM

Still in some embodiments, ID-8 is at a concentration of about 1 μM to about 5 μM and FK-506 is at a concentration of about 1 nM to about 2 nM.

In some embodiments, the serum-free medium is essentially devoid of animal contaminants.

In some embodiments, the serum-free medium is essentially devoid of human contaminants.

In some embodiments, the serum-free medium is essentially devoid of any antibiotic drug.

Another aspect of the present disclosure provides a method of producing a cultured meat by culturing cells in any of the herein disclosed cell culture medium and producing a cultured meat from the cultured cells. Further aspect of the present disclosure provides a cultured meat produced by the methods disclosed herein.

In some embodiments, the cells are from edible animals. In some embodiments, the source of the cells is any edible species desired for consumption, which include, but are not limited to, livestock, game, poultry, fish, shellfish, crustaceans, and mollusk.

In some embodiments, the source of the cells is a livestock, e.g., cattle, sheep, pig, goat, lamb, horse, donkey, rabbit, and mule. In some embodiments, the source of the cells is an animal traditionally considered “game”, e.g., caribou, bear, boar, deer, elk, and moose. In some embodiments, the source of the cells is a poultry, e.g., chicken, duck, goose, guinea fowl, quail, and turkey. In some embodiments, the source of the cells is a fish, e.g., bass, carp, catfish, Chilean sea bass, cod, flounder, halibut, mahi mahi, monkfish, pike, perch, orange roughy, salmon, shad, snapper, swordfish, tilapia, trout, and tuna. In some embodiments, the source of the cells is a crustacean, e.g., crab, crayfish, lobster, prawn, and shrimp. In some embodiments, the source of the cells is a mollusk, e.g., clams, mussels, octopus, oysters, scallops, and squid.

In some embodiments, the method comprises cultured cells wherein the cells are fibroblasts. The fibroblasts can be from an edible animal. In an embodiment, the fibroblasts are bovine fibroblasts or chicken fibroblasts.

Chicken embryonic fibroblasts are widely used for the production of viruses and vaccines. Together with chicken embryonic liver cells, they are produced from specific pathogen-free (SPF) embryos and sold by Charles River Laboratories (Wilmington, Mass.) and other companies. While chicken liver cells show limited proliferation in culture, like their mammalian counterparts, chicken fibroblasts can undergo over 30 population doublings, producing about 2.6 ton of cells before spontaneously immortalizing without becoming tumorigenic. Spontaneously transformed chicken fibroblasts, such as UMNSAH/DF-1 (CRL-12203), can be bought directly from ATTC (Manassas, Va.). While the growth potential of fibroblast is excellent, the cells primarily form inedible connective tissue.

Chicken embryonic endothelium can be easily isolated but their growth potential is unknown and can be organ specific. Mouse endothelial cells can undergo 30 population doublings, while human endothelial cells seldom pass 12 population doublings. Chicken embryonic muscle cells (myocytes) can be similarly isolated but have a very limited growth potential. Mouse and human muscle cells seldom pass 12 population doublings. Myogenesis, the formation of new muscle tissue, is uncommon past the neonatal stage of life in most species. Small molecules of the present disclosure can conceptually be used to modulate this behavior.

Numerous groups produced chicken embryonic stem cells (cESC) over the last decade. Cells are isolated from fertilized chicken eggs and are essentially immortal. Chicken induced pluripotent stem cells (ciPSC) were produced from quail embryonic fibroblasts by reprogramming factors OCT4, NANOG, SOX2, LIN28, KLF4, and C-MYC and more recently chicken fibroblasts using OCT4, KLF4, and C-MYC. Cells are essentially immortal but are genetically engineered.

Recently, mouse pluripotent stem cells were induced from fibroblasts using small molecules permitting the differentiation of multiple cell types, including myocytes, hepatocytes, and endothelial cells as well as complex embryoid bodies. Chemical induction of ciPSC offers an alternative approach to convert fibroblasts to other cell types.

Chemical compounds offer an attractive alternative to growth factors and genetic engineering that are generally used to support cell growth, or to switch one cell type to another through reprogramming or differentiation. Small molecules are less expensive, have lower lot-to-lot variability, and are non-immunogenic and are much more stable. In one study, a high content screen was used to identify FPH1 and FPH2, small molecules that promoted proliferation of primary human hepatocytes (Hou et al., Science, 341(6146): 651-654, 2013). This approach is appealing, as small molecules could replace growth factors in serum-free medium formulations, dramatically reducing costs while increasing safety.

In a more recent study, a combination of nine compounds that induced human fibroblasts to turn into cardiomyocytes were identified (Shan et al., Nature Chemical Biology, 9: 514-520, 2013), while others used a seven compound combination to transform mouse fibroblast cells (Cao et al., Science, 352(6290): 1216-1220, 2016). Considering that many of the signaling pathways are conserved among different animals, a relatively similar combination could be used to transform chicken fibroblasts into myocytes.

As mentioned above, cell culture medium often contains fetal bovine serum (FBS) that provides attachment factors, fatty acids, growth factors, hormones, and albumin. FBS can usually be replaced with serum replacement (e.g. KO-serum) that is composed of amino acids, vitamins, and trace elements in addition to transferrin, insulin, and lipid-rich bovine serum albumin. While both transferrin and insulin are produced in bacteria using recombinant technology, albumin is usually animal derived. However, plant and bacteria-derived recombinant human albumin (e.g. Cellastim™) are available through several companies, including Sigma-Aldrich (St. Louis, Mo.).

Chicken fibroblast medium is traditionally composed of Ml 99 medium supplemented with 10% FBS, tryptose phosphate and glutamine. However, serum-free medium for the growth of mammalian fibroblasts is now readily available. Medium is composed of M199 supplemented with 0.5 mg/mL albumin, 0.6 μM linoleic acid, 0.6 μg/mL lecithin, 5 ng/mL bFGF, 5 ng/mL EGF, 30 μg/mL TGFpi, 7.5 mM glutamine, 1 μg/mL hydrocortisone, 50 μg/mL ascorbic acid, and 5 μg/mL insulin. This medium PCS-201-040 is available from ATCC (Manassas, Va.) and is reported to support 4-fold faster proliferation of human fibroblasts. Chicken hepatocytes are similarly supported by a serum-free culture medium designed for human and mouse hepatocytes. Medium is composed of Williams E basal medium supplemented with albumin, insulin, transferrin, and hydrocortisone.

Perfused culture medium can also include an oxygen carrier. Hemoglobin based oxygen carriers include hemoglobin derivatives either recombinant or chemically modified, encapsulated hemoglobin or modified (e.g. cross-linked) red blood cells. Alternatives include Perfluorocarbon based alternatives such as those developed in Nahmias et al. (The FASEB Journal, 20(14): 2531-2533).

It should be noted that normally, primary fibroblast cells are capable of a limited cell division, and thus undergo cellular senescence after about 30 population doublings (e.g., 10 passages). Methods of generating immortalized fibroblastoid cell lines include genetic manipulation by introduction of a telomerase gene, or SV40, or HPVE6/E7 gene using known methods.

It is contemplated that other avian fibroblast cells are also suitable, e.g., duck, goose, and quail fibroblast cells.

Yet another aspect of the present disclosure provides a method of growing cells in vitro by culturing cells in any of the herein disclosed cell culture medium.

Further aspect of the present invention provides use of one or more FGF activators in a cell culture medium essentially devoid of any protein-based growth factors excluding peptide-based hormones or steroid-based hormones.

It is contemplated that the media, e.g., serum replacement, media supplement, complete media, described herein is useful for culture of cells in vitro, especially for cells that typically require serum supplements or defined media for adequate growth in vitro. Such cells include eukaryotic cells, such as mammalian cells, and insect cells. Mammalian cells contemplated to benefit from use of the serum replacement, complete media or media supplement include, without limitation, hamster, monkey, chimpanzee, dog, cat, cow/bull, pig, mouse, rat, rabbit, sheep and human cells. Insect cells include cells derived from Spodoptera frugiperda (caterpillar), Aedes aegypti (mosquito), Aedes albopictus (mosquito), Drosophila melanogaster (fruitfly), and Bombyx mori.

It is contemplated that the cells cultured with the serum replacement, complete media or media supplement, are immortalized cells (a cell line) or non-immortalized (primary or secondary) cells, and can be any of a wide variety of cell types that are found in vivo. Exemplary cell types include, but are not limited to, fibroblasts, keratinocytes, epithelial cells, ovary cells, endothelial cells, glial cells, neural cells, formed elements of the blood (e.g., lymphocytes, bone marrow cells), chondrocytes and other bone-derived cells, hepatocytes, pancreas cells, and precursors of these somatic cell types.

In some embodiments, the cells contemplated for use with the media disclosed above and herein are isolated from a mammalian subject. Cells isolated from a mammalian subject include, but are not limited to, pluripotent stem cells, embryonic stem cells, bone marrow stromal cells, hematopoietic progenitor cells, lymphoid stem cells, myeloid stem cells, lymphocytes, T cells, B cells, macrophages, endothelial cells, glial cells, neural cells, chondrocytes and other bone-derived cells, hepatocytes, pancreas cells, precursors of somatic cell types, and any carcinoma or tumor derived cell.

In some embodiments, the cells are a cell line. Exemplary cell lines include, but are not limited to, Chinese hamster ovary cells, including CHOK1, DXB-11, DG-44, and CHO/-DHFR; monkey kidney CV1, COS-7; human embryonic kidney (HEK) 293; baby hamster kidney cells (BHK); mouse sertoli cells (TM4); African green monkey kidney cells (VERO); human cervical carcinoma cells (HELA); canine kidney cells (MDCK); buffalo rat liver cells (BRL 3A); human lung cells (W138); human hepatoma cells (Hep G2; SK-Hep); mouse mammary tumor (MMT); TRI cells; MRC 5 cells; FS4 cells; a T cell line (Jurkat), a B cell line, mouse 3T3, RIN, A549, PC12, K562, PER.C6®, SP2/0, NS-0, U20S, HT1080, L929, hybridomas, tumor cells, and immortalized primary cells.

Exemplary insect cell lines, include, but are not limited to, Sf9, Sf21, HIGH FIVE™, EXPRESSF+®, S2, Tn5, TN-368, BmN, Schneider 2, D2, C6/36 and KC cells.

Additional cell types and cell lines are disclosed in WO 2006/004728, herein incorporated by reference. These cells include, but are not limited to, CD34+ hematopoietic cells and cells of myeloid lineage, 293 embryonic kidney cells, A-549, Jurkat, Namalwa, Hela, 293BHK cells, HeLa cervical epithelial cells, PER-C6 retinal cells (PER.C6), MDBK (NBL-I) cells, 911 cells, CRFK cells, MDCK cells, BeWo cells, Chang cells, Detroit 562 cells, HeLa 229 cells, HeLa S3 cells, Hep-G2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-I cells, Y-I cells, LLC-PK1 cells, PK (15) cells, GH1 cells, GH3 cells, L2 cells, LLC-RC 256 cells, MH1C1 cells, XC cells, MDOK cells, VSW cells, TH-I, B1 cells, or derivatives thereof, fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, fibroblast and fibroblast-like cell lines), TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, MR-90 cells, MRC-5 cells, WI-38 cells, WI-26 cells, MiC11 cells, CV-I cells, COS-I cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-I1 cells, NOR-IO cells, C3H/IOTI/2 cells, HSDM1C3 cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, NSO, NS1, Swiss/3T3 cells, Indian muntjac cells, SIRC cells, Cn cells, Jensen cells, COS cells and Sp2/0 cells, Mimic cells and/or derivatives thereof.

Cell culture conditions contemplated herein may be adapted to any culture substrate suitable for growing cells. Substrates having a suitable surface include tissue culture wells, culture flasks, roller bottles, gas-permeable containers, flat or parallel plate bioreactors or cell factories. Also contemplated are culture conditions in which the cells are attached to microcarriers or particles kept in suspension in stirred tank vessels.

Cell culture methods are described generally in the Culture of Animal Cells: A Manual of Basic Technique, 6th Edition, 2010 (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other reference texts include Creating a High Performance Culture (Aroselli, Hu. Res. Dev. Pr. 1996) and Limits to Growth (D. H. Meadows et al., Universe Publ. 1974). Tissue culture supplies and reagents are well-known to one of skill and are commercially available.

It is understood that the cells are placed in culture at densities appropriate for the particular cell line or isolated cell type used with the serum replacement, complete media or media supplement. In certain embodiments the cells are cultured at 1×10³, 5×10³, 1×10⁴, 5×10⁴, 1×10⁵, 5×10⁵, 1×10⁶ or 5×10⁶ cells/ml.

Additional components in the cell culture media are also contemplated. In some embodiments, the cell culture medium may comprise one or more elements of a base medium and supplements as described herein, e.g., salts, amino acids, vitamins, buffers, nucleotides, antibiotics, trace elements, antioxidants and glucose or an equivalent energy source, such that the cell culture medium is capable of be used as a serum-free complete medium.

Exemplary inorganic salts include, but are not limited to, potassium phosphate, calcium chloride (anhydrous), cupric sulfate, ferric nitrate, ferric sulfate, magnesium chloride (anhydrous), magnesium sulfate (anhydrous), potassium chloride, sodium bicarbonate, sodium chloride, sodium phosphate dibasic anhydrous, sodium phosphate monobasic, tin chloride and zinc sulfate. Exemplary organic salts include, but are not limited to, sodium bicarbonate or HEPES.

Exemplary sugars include, but are not limited to, dextrose, glucose, lactose, galactose, fructose and multimers of these sugars.

Exemplary antioxidants include, but are not limited to tocopherols, tocotrienols, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), flavonoids, isoflavones, lycopene, beta-carotene, selenium, ubiquinone, luetin, S-adenosylmethionine, glutathione, taurine, N-acetylcysteine, citric acid, L-carnitine, BHT, monothioglycerol, ascorbic acid, propyl gallate, methionine, cysteine, homocysteine, gluthatione, cystamine and cysstathionine, and glycine-glycine-histidine (tripeptide).

Exemplary trace elements, include, but are not limited to, copper, iron, zinc, manganese, silicon, molybdate, molybdenum, vanadium, nickel, tin, aluminum, silver, barium, bromine, cadmium, cobalt, chromium, calcium, divalent cations, fluorine, germanium, iodine, rubidium, zirconium, or selenium. Additional trace metals are disclosed in WO 2006/004728.

In some embodiments, the media or liquid base mix comprises an iron source or iron transporter. Exemplary iron sources include, but are not limited to, ferric and ferrous salts such as ferrous sulfate, ferrous citrate, ferric citrate, ferric nitrate, ferric sulfate, ferric ammonium compounds, such as ferric ammonium citrate, ferric ammonium oxalate, ferric ammonium fumarate, ferric ammonium malate and ferric ammonium succinate. Exemplary iron transporters include, but are not limited to, transferrin and lactoferrin.

In some embodiments, the media or liquid base mix may comprise one or more elements of a base media and supplements as described above, e.g., salts, amino acids, vitamins, buffers, nucleotides, antibiotics, trace elements, antioxidants and glucose or an equivalent energy source, such that the media is capable of use as a serum-free complete media.

In some embodiments, the media or liquid base mix may further comprise a copper source or copper transporter (e.g., GHK-Cu). Exemplary copper sources include, but are not limited to, copper chloride and copper sulfate.

In some embodiments, the iron source or copper source is added to a serum replacement media at a final concentration in the range of about 0.05 to 250 ng/ml, 0.05 to 100 ng/ml, from about 0.05 to 50 ng/ml, from about 0.05 to 10 ng/ml, from about 0.1 to 5 ng/ml, from about 0.5 to 2.5 ng/ml, or from about 1 to 5 ng/ml. It is further contemplated that the iron source or copper source is in a final concentration in the serum replacement of about 0.05, 0.1, 0.25, 0.35, 0.45, 0.5, 0.6, 0.7, 0.8, 1, 1.5, 2, 2.5, 3, 4, 5, 6, 7, 8, 9, or 10 ng/ml.

In some embodiments, the serum replacement or media supplement is added to a basic media. Standard basic media are known in the field of cell culture and commercially available. Examples of basic media include, but are not limited to, Dulbecco's Modified Eagle's Medium (DMEM), DMEM F12 (1:1), Iscove's Modified Dulbecco's Medium, Ham's Nutrient mixture F-10 or F-12, Roswell Park Memorial Institute Medium (RPMI), MCDB 131, Click's medium, McCoy's 5A Medium, Medium 199, William's Medium E, and insect media such as Grace's medium and TNM-FH.

The serum replacement and media supplement described herein are also contemplated for use in commercially available serum-free culture media. Exemplary serum-free medias, include but are not limited to, AIM-V (Life Technologies, Carlsbad, Calif.), PER-C6 (Life Technologies, Carlsbad, Calif.), Knock-Out™ (Life Technologies), StemPro® (Life Technologies), CellGro® (Corning Life Sciences—Mediatech Inc., Manassas, Va.).

Any of these media are optionally supplemented with salts (such as sodium chloride, calcium, magnesium, and phosphate), amino acids, vitamins, buffers (such as HEPES), nucleotides (such as adenosine and thymidine), antibiotics (such as gentamicin drug), trace elements (defined as inorganic compounds usually present at final concentrations in the micromolar range), antioxidants and glucose or an equivalent energy source. Any other necessary supplements may also be included at appropriate concentrations that would be known to those skilled in the art. The culture conditions, such as temperature, pH, and the like, will be apparent to the ordinarily skilled artisan.

It is contemplated that the medium compositions are packaged in unit forms. In one embodiment, the medium (serum replacement, medium supplement, complete media or cryopreservation media) is packaged in a volume of 10 ml, 50 ml, 100 ml, 500 ml or 1 L.

The disclosure further provides for a kit comprising a cell culture medium as described above and herein and instructions for use. In some embodiments, the medium is packaged in a container with a label affixed to the container or included in the package that describes use of the compositions for use in vitro, in vivo, or ex vivo. Exemplary containers include, but are not limited to, a vessel, vial, tube, ampoule, bottle, flask, and the like. It is further contemplated that the container is adapted for packaging the medium, e.g., serum replacement or media supplement in liquid or frozen form. It is contemplated that the container is made from material well-known in the art, including, but not limited to, glass, polypropylene, polystyrene, and other plastics. In some embodiments, the compositions are packaged in a unit dosage form. The kit optionally includes a device suitable for combining the serum replacement or medium supplement with a basic medium. In some embodiments, the kit contains a label and/or instructions that describes use of the medium for cell culturing or cryopreservation.

All applications and all documents cited herein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The following examples are offered by way of illustration and not by way of limitation.

### Example 1: Effects of ID-8 and/or FK-506 on Bovine Fibroblast Cell Growth

Bovine anchorage-independent fibroblasts were differentiated into anchorage-independent adipocytes by standard differentiation protocols. FMT-SBF-1 (bovine non-adherent fibroblasts) were grown in adipogenesis medium containing 200 μM oleic acid together with a PPARgamma agonist. A synthetic inhibitor (Rosiglitazone) and a natural inhibitor (Pristanic acid) were both tested.

Effects of ID-8 and/or FK-506 (alone or combined) on bovine fibroblast cell growth were evaluated. Bovine fibroblasts adapted to suspension culture were seeded in 0.3 millions/ml in a total volume of 20 ml in cell culture flasks. The flasks were kept in a shaker incubator with 100 rpm, 37° C., and 5% CO₂. Cell counts were done on Day 3 using automatic cell counter (Cellaca®) with APOI staining to eliminate the dead cells from the counts.

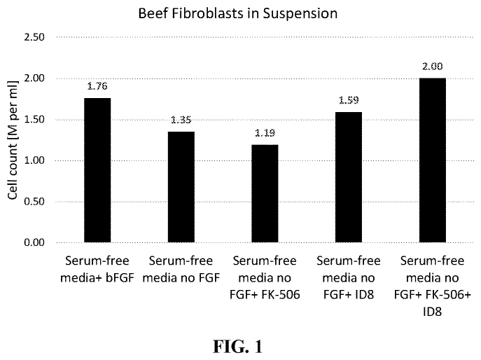

The results are shown in FIG. 1. ID-8 alone (1 μM) obtained a comparable effect on bovine fibroblast cell growth as compared to FGF (10 ng/ml), which suggests that ID-8 can at least partially replace FGF in cell culture media. FK-506 alone (2 nM) didn't obtain a comparable effect on bovine fibroblast cell growth as compared to FGF (10 ng/ml). It is noted that FK-506 was tested at a much lower concentration than ID-8 in this study. The results further show that the combination of both small molecules, ID-8 (1 μM) and FK-506 (2 nM), exhibited the maximum cell growth after three days of culturing when compared to the FGF control (10 ng/ml). It is anticipated that the concentrations of ID-8 and FK-506 can vary in the ranges of about 0.5 μM to about 10 μM and of about 2 nM to about 10 nM, respectively. This study suggests that the combination of both ID-8 and FK-506 can completely replace FGF in serum-free culture media for bovine fibroblasts.

### Example 2: Effects of ID-8 and/or FK-506 on Chicken Fibroblast Cell Growth

Chicken anchorage-independent fibroblasts were differentiated into anchorage-independent adipocytes by standard differentiation protocols. FMT-SCF-2 (chicken non-adherent fibroblasts) were grown in adipogenesis medium containing 200 μM oleic acid together with a PPARgamma agonist. A synthetic inhibitor (Rosiglitazone) and a natural inhibitor (Pristanic acid) were both tested.

Effects of ID-8 and FK-506 on chicken fibroblast cell growth were evaluated. Chicken fibroblasts adapted to suspension culture were seeded in 0.3 millions/ml in a total volume of 20 ml in cell culture flasks. The flasks were kept in a shaker incubator with 100 rpm, 39° C., and 5% CO₂. Cell counts were done on Day 3 using automatic cell counter (Cellaca®) with APOI staining to eliminate the dead cells from the counts.

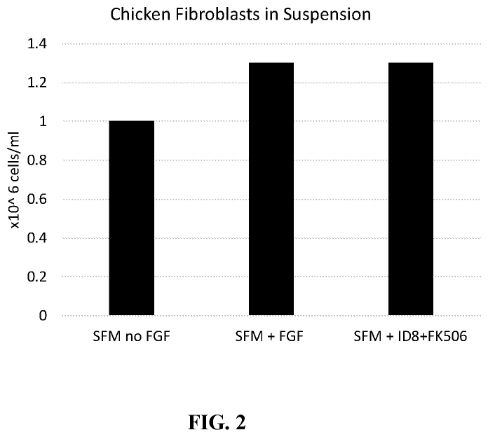

The results are shown in FIG. 2. The combination of ID-8 (0.5 μM) and FK-506 (2 nM) showed similar effects on cell growth when compared to the FGF control (10 ng/ml). It is anticipated that the concentrations of ID-8 and FK-506 can vary in the ranges of about 0.5 μM to about 10 μM and of about 2 nM to about 10 nM, respectively. This study suggests that the combination of both ID-8 and FK-506 can completely replace FGF in serum-free culture media for chicken fibroblasts.

### Example 3: Effects of Gradient Concentrations of ID-8 and FK-506 on Ovine Fibroblasts

The effects of gradient concentration of both small molecules (ID-8 and FK-506) in ranges from about 1-50 μM for ID-8 and about 1-50 nM for FK-506 were tested on ovine fibroblasts 2D culture. Ovine fibroblasts were seeded in 96-well plate at a density of 1000 cells/well. ID-8 and FK-506 in serum free medium were incubated with the fibroblast cells for 48 hours before adding XTT (cell proliferation assay from Biological Industries, Israel) according to company protocol. Colorimetric signal was measured after 8 hours of XTT addition. Blank (medium only) measurements were subtracted from the sample measurements (n=4).

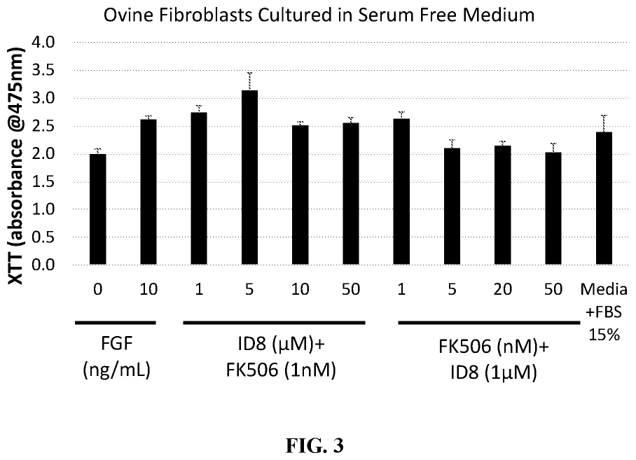

The results show that the best cell growth was obtained when FK-506 was at the concentration of about 1-2 nM and ID-8 was at the concentration of about 1-5 μM (the optimal concentrations) (FIG. 3). Higher concentrations of ID-8 (e.g., 10 μM or 50 μM) still showed more cell growth than the control (no FGF). On the other hand, higher concentration of FK-506 (e.g., 5 nM, 20 nM, or 50 nM) did not show significant effect on cell growth even with ID-8 at the optimal concentration of 1 μM.

Although the present invention and its advantages have been described in detail, it should be understood that various changes, substitutions and alterations can be made herein without departing from the spirit and scope of the invention as defined in the appended claims.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A cell culture medium for culturing cells from edible animals, said medium comprising a serum-free medium and one or more fibroblast growth factor (FGF) activators, wherein said one or more FGF activators include an indole derivative, a macrolide antibiotic, or a combination thereof.

2. The cell culture medium of claim 1, wherein said medium comprises less than 1 ng/ml of FGF, epidermal growth factor (EGF), transforming growth factor-β (TGF-β), or a combination thereof.

3. The cell culture medium of claim 1, wherein said medium is essentially devoid of any protein-based growth factors, wherein said protein-based growth factors exclude peptide-based hormones or steroid-based hormones.

4-6. (canceled)

7. The cell culture medium of claim 1, wherein the indole derivative is ID-8.

8. (canceled)

9. The cell culture medium of claim 1, wherein the macrolide antibiotic is Tacrolimus (FK-506).

10. The cell culture medium of claim 1, wherein the one or more FGF activators include ID-8 and FK-506.

11. The cell culture medium of claim 7, wherein ID-8 is at a concentration of about 0.5 μM to about 50 μM.

12. (canceled)

13. The cell culture medium of claim 9, wherein FK-506 is at a concentration of about 1 nM to about 20 nM.

14. (canceled)

15. The cell culture medium of claim 10, wherein ID-8 is at a concentration of about 0.5 μM to about 50 μM and FK-506 is at a concentration of about 1 nM to about 20 nM.

16. The cell culture medium of claim 15, wherein ID-8 is at a concentration of about 1 μM to about 10 μM and FK-506 is at a concentration of about 1 nM to about 2 nM.

17-25. (canceled)

26. A kit comprising the cell culture medium of claim 1 and instructions for use.

27. A method of producing a cultured meat, comprising culturing cells from edible animals in the cell culture medium of claim 1, and producing a cultured meat from the cultured cells.

28. (canceled)

29. The method of claim 27, wherein the edible animal is livestock, game, poultry, fish, crustaceans, and mollusk.

30. The method of claim 27, wherein the cells comprise fibroblasts.

31. The method of claim 30, wherein the fibroblasts are bovine fibroblasts or chicken fibroblasts or ovine fibroblasts.

32. A cultured meat produced by the method of claim 27.

33. The method of claim 27, wherein the cells are cultured as single cell suspensions.

34. The method of claim 33, wherein the cells comprise CHO cells or EB66 cells.

35. A method of growing cells from edible animals in vitro, comprising culturing the cells in the cell culture medium of claim 1.

36. A method of culturing cells from edible animals comprising incubating cells in a cell culture medium comprising one or more FGF activators, wherein said one or more FGF activators include an indole derivative, a macrolide antibiotic, or a combination thereof, and wherein said cell culture medium is essentially devoid of any protein-based growth factors, wherein said protein-based growth factors exclude peptide-based hormones or steroid-based hormones.
